(19) United States
(12) Patent Application Publication
(10) Pub. No.: US 2010/0284871 A1
(43) Pub. Date: Nov. 11, 2010

(54) Environmental contaminant removal solution and system for delivery

(76) Inventor: James W. Thomas, Jacksonville, FL (US)

Correspondence Address: James W. Thomas, 185 8th Avenue South, Jacksonville, FL, 32250, US

(21) Appl. No.: 12/387,746

(22) Filed: May 8, 2009

(51) Int. Cl. A61L 2/20; A01N 31/08; A01P 1/00

(52) U.S. Cl. 422/292; 514/737

(57) ABSTRACT

An aqueous solution effective in preventing the spread and contamination effects of non-enveloped virus, bacteria, molds and spores is disclosed. The solution includes between 0.18-0.22, and preferably, 0.22 percent by weight of ortho-benzyl-para chlorophenol; between 0.22-0.26, and preferably, 0.26 percent by weight of ortho phenylphenol; between 0.06-0.10, and preferably, 0.10 percent by weight of para-tertiary amylphenol; between 0.14-0.16, and preferably, 0.16 percent by weight of isopropanol; between 0.08-0.10, and preferably 0.10 percent by weight of sodium hydroxide and between 0.08-0.10, and preferably, 0.10 percent by weight of ethylene glycol. Distilled water is used as the solvent at a ratio of ½ oz of concentrate to one gallon of distilled water. In this manner, the effects of impurities in the solvent are substantially reduced or eliminated.

### FIELD OF THE INVENTION

The present invention generally relates to the field of environmental contamination removal and, more particularly, to an aqueous solution capable of controlling and/or eliminating environmental contaminants and a system for delivering the solution within a structure.

### BACKGROUND OF THE INVENTION

Airborne contaminants, for example, bacteria, molds, spores, animal dander and other contaminants and virus strands, for example, the avian flu and other airborne contaminants and combinations thereof have become more prevalent and dangerous in the past several years. In particular, the avian flu virus and analogous virus strands can cause serious health problems to both humans and animals, alike that come into direct or indirect contact with such viruses. To date, a cure for the effects of the avian flu virus, or a method of preventing the spread of the virus, has not been developed.

Phenols, for example, chloropenol, phenyl phenol and amyl phenol are often used as disinfectants that are effective against enveloped viruses and spores. Pine-sol™ is a common type of phenol that is used as a disinfectant. A drawback associated with phenols is that their application is typically limited to being directly applied to a surface or area of interest, for example, a counter top. Thus, applying phenols over a large area, for example, a home, office building, airport and other larger structures will at least be time consuming and often times difficult. Another drawback associated with phenols is that at concentrations greater than about two percent, they are highly toxic to animals, and in particular to cats.

Other substances, for example, carboxylic acid and carboxylic salts have been used to protect fruits, vegetables and other produce from spoiling; thereby, increasing their shelf life. However, to date, carboxylic acids and carboxylic salts alone, or in combination, have not proven to provide an adequate defense against the effects of non-enveloped or airborne viruses. Additionally, carboxylic acid and carboxylic salts have not typically used to prevent the spread or effects of airborne viruses, bacteria, molds and/or spores within physical structures.

### SUMMARY OF THE INVENTION

The present invention is directed to an aqueous solution that is composed, for example, of between 0.18-0.22 percent by weight of ortho-benzyl-para chlorophenol; between 0.22-0.26 percent by weight of ortho phenylphenol; between 0.06-0.10 percent by weight of para-tertiary amylphenol; between 0.14-0.16 percent by weight of isopropanol; between 0.08-0.10 percent by weight of sodium hydroxide and between 0.08-0.10 percent by weight of ethylene glycol. The aqueous solution is diluted in distilled water from a concentrate at a ratio of one-half (½ oz.) of concentrate per one gallon of distilled water (1:256). Distilled water is used as the solvent, as opposed to other types of water, for example, water out of a standard tap or ground water, due to the high purity of distilled water. The higher the level of impurities in the solvent the less effective the aqueous solution is in preventing the effects and spread of influenza and other airborne viruses.

The inventors have found through experimentation that the aforementioned aqueous solution is effective in substantially reducing or eliminating the spread and effects of the avian flu virus and other strands of influenza from within large surface areas, for example, homes, office building and other large structures. In an exemplary embodiment, the aqueous solution is introduced into a building as a fog. In application, the aqueous solution is turned into a fog via a suitable fog generation device. The resulting fog is then provided to the heating, ventilation and air condition (HVAC) system of the structure, which results in the solution being transmitted throughout the building.

An advantage provided by the present invention is that is substantially reduces or eliminates the transmission and effects of non-enveloped viruses.

Another advantage provided by the present invention is that the solution is easily applied throughout large structures, and not limited to specific areas.

A feature of the present invention is that the solution is portable and not harmful to humans or animals.

### BRIEF DESCRIPTION OF THE DRAWINGS

The aforementioned and related advantages and features of the present invention will be best appreciated and understood upon review of the following detailed description of the invention, taken in conjunction with the following drawings, where like numerals represent like elements, in which:

FIG. 1 is a schematic block diagram of a structure, including a heating, ventilation and air conditioning system configured to disperse the aqueous solution of the present invention throughout a structure; and

FIG. 2 is an exploded view of the components that convert the aqueous solution of the invention into a fog for dispersal through the heating, ventilation and air conditioning system illustrated in FIG. 1.

### DETAILED DESCRIPTION OF THE INVENTION

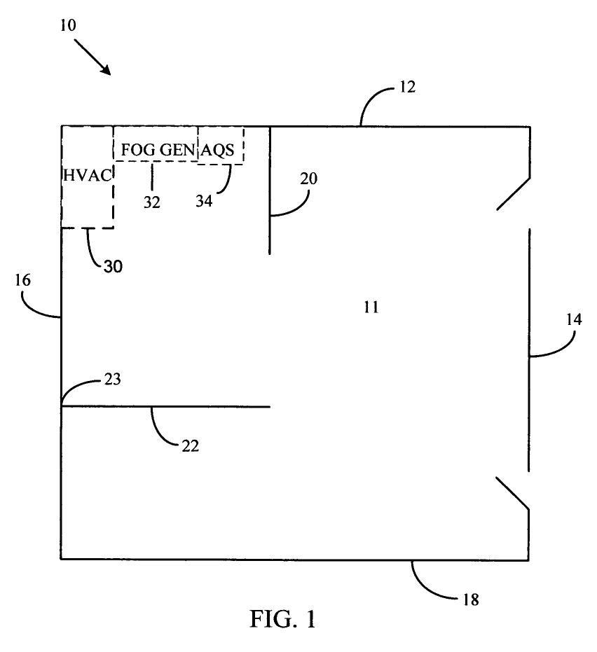

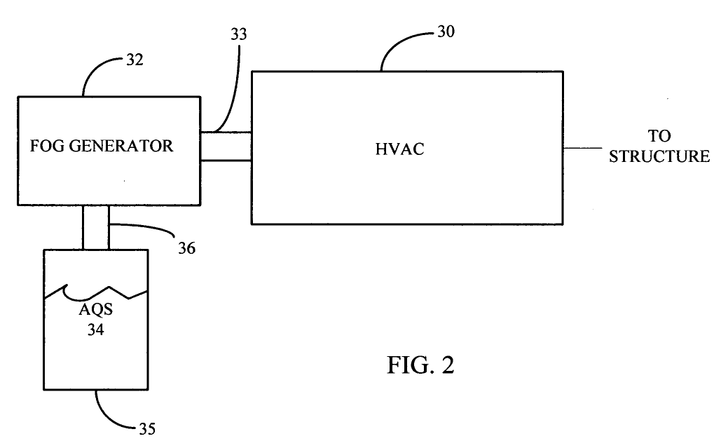

An exemplary embodiment of the present invention will now be described with reference to FIGS. 1-2. Airborne contaminants and/or non-enveloped viruses, for example, the avian flu virus, the several strands of the influenza virus, bacteria, molds and spores often times lodge themselves within the walls of a building or other hard to reach areas such as cracks or crevices where walls intersect or where windows or other openings couple to walls. Controlling or eliminating contaminants from these areas have heretofore been extremely difficult to accomplish due to the spot application nature of currently used disinfectants. Introducing disinfectants into small surface areas, for example, cracks within a wall or other openings cannot be efficiently accomplished using liquid spot treatments. For those chemicals that can be made into a mist, the use of aerosols, which provides the courier for those chemicals, has its own well-documented drawbacks. The present invention overcomes such difficulties by provided an anti-contamination product within a large surface area, including those hard to reach area of structures as a fog, both during and after construction, to prevent the negative effects of airborne viruses, molds, spores and other contaminants.

FIG. 1 is a schematic block diagram of a structure 10, for example, a home, office building, airport, storage facility or other suitable building or structure, having an internal area 11 bounded by exterior walls 12-16 and including at least two internal walls 20, 22. The internal walls 20, 22 may be used to partition the internal area 11 into a series of segments, for example, cubicles or to separate the segments of the structure 10 based on use, for example, one region may be used to maintain a customer service counter, while the remaining area are used as a customer waiting area or other such use. The structure 10 includes a heating, ventilation and air condition (HVAC) system 30, configured to both control the temperature of the structure 10 and to disperse the aqueous solution (AQS) 34 into the environment 11 of the structure 10 according to the present invention.

A fog generation system 32 is coupled between the HVAC system 30 and the aqueous solution 34. The fog generation system 32 is operative to convert the aqueous solution 34 into a fog that is then provided to the HVAC 30 for dispersal within the internal area 11 through the HVAC system 30 of the structure 10. Also shown, is an intersection point 23 were one of the internal walls 22 intersects an outer wall 16. It is typically at such an intersection point 23 where mold, spores and other airborne contaminants are located, and are difficult to remove given the small surface area typically associated with such intersection points.

The aqueous solution 34 is comprised of about 26 percent by weight of phenol compounds, for example, ortho benzyl-para chlorophenol, ortho phenylphenol, and para-tertiary amylphenol; less than about eight percent by weight of isopropanol; less than about five percent by weight of sodium hydroxide; and less than about five percent by weight of ethylene glycol. The isopropanol and ethylene glycol are used to prevent the phenols from precipitating during any storage periods. The sodium hydroxide is used to make the phenolic salts, which make the resolution soluble in water. In a preferred embodiment, the aqueous solution 34 is composed of, for example, between 0.18-0.22, and preferably, 0.22 percent by weight of ortho-benzyl-para chlorophenol; between 0.22-0.26, and preferably, 0.26 percent by weight of ortho phenylphenol; between 0.06-0.10, and preferably, 0.10 percent by weight of para-tertiary amylphenol; between 0.14-0.16, and preferably, 0.16 percent by weight of isopropanol; between 0.08-0.10, and preferably 0.10 percent by weight of sodium hydroxide and between 0.08-0.10, and preferably, 0.10 percent by weight of ethylene glycol.

The aqueous solution is diluted with distilled water from a concentrate at a ratio of one-half ounce (½ oz) of concentrate per one gallon of distilled water—a 1:256 ratio. Distilled water is used as the solvent, as opposed to other types of water, for example, water out of a standard tap, spring water or ground water due to the high purity of distilled water. The higher the level of impurities present in the solvent, the less effective the resulting aqueous solution is in preventing the effects and spread of influenza and other non-enveloped and airborne viruses.

The aqueous solution 34 may be applied to a structure 10 either during the construction initial construction process before the walls have been completed, or after construction. During initial construction period, the aqueous solution 34 can be applied to the area between the internal walls 20, 22 and the exterior walls 12-16 after the corresponding walls are erected, but before any insulation, sound proofing or other materials are placed into the walls and the walls are enclosed, for example, with sheet rock or other suitable material. The aqueous solution 34 may be applied as either a spray from a suitable spray application device or from a fog generation machine, capable of turning the aqueous solution 34 into a fog that is absorbed into the walls and the small areas between the walls and where, for example, the interior walls 20, 22 and the exterior walls 12-16 intersect. After the walls are filled in and finished a subsequent treatment may be provided to further enhance the effects of the aqueous solution. An advantage associated with treating a structure before it is completed, is that the skeleton materials of the structure may be treated; thereby, preventing any airborne, non-enveloped or harmful viruses, molds, spores and other suitable contaminants from affecting the structure of the building at its foundation.

In post construction structures, for example, the structure 10 illustrated in FIG. 1, the aqueous solution 34 may be provided into the environment (e.g. internal area 11) of the structure through the HVAC system 30. In this way, the atmospheric environment as well as the structural environment of the structure 10 may be protected against the transmission and harmful effects of airborne contaminants. Referring to FIG. 2, illustrated therein is an exploded view of the HVAC system 30 and the components used to provide the aqueous solution 34 to the HVAC system 30. The aqueous solution 34 is maintained within one or more containers 35, having sufficient volume to store enough solution for the portion of the structure 10 associated with a particular HVAC 30, if there are more than one HVAC components within or servicing the structure 10 or the entire structure 10 if there is only one HVAC component. FIGS. 1-2 illustrate a structure having a single HVAC 30 component; however, those of ordinary skill in the art of heating, ventilation and cooling will appreciate and recognize that more than one HVAC system may be used to provide ventilation within a structure and such configuration are contemplated by and fall within the spirit and scope of the present invention.

As shown, the container 35 holding the aqueous solution 34 is coupled to a suitable fog generation device 32, via a conduit 36. In application, the aqueous solution 34 is provided to the fog generation device 32, which, in turn, converts the aqueous solution 34 into a fog 33. The fog 33 is provided to the HVAC system 30, which carries the product into the atmosphere of the structure 10. The fog generation device 32 provides the fog 33 at set intervals and amounts in order to maintain the level of effectiveness against bacteria, viruses and mold constant throughout the structure 10. The dispersal and amount of solution provided to the HVAC system 30 will vary depending on the size (e.g. square footage) of the structure being treated that the particular HVAC system 30 covers. For example, a structure 10 having a surface area of approximately 10,000 ft² would require approximately ten gallons of solution for an initial treatment, and between two and five gallons of the aqueous solution provided into the atmosphere (e.g. internal area 11) every two to three days depending on the size of the HVAC unit being used, the overall humidity conditions in the geographic region of the structure 10 and the time of year.

As the aqueous solution 34 is provided into the structure 10 through the HVAC system 30, no special devices or treatments are required to introduce the aqueous solution 34 into the atmosphere. Additionally, as the solution 34 is introduced as a fog, direct application to spot or specific areas within the structure 10 are not required. Moreover, as the solution 34 is provided as a fog within the structure, the solution 34 will reach those areas within the structure 10, for example, the wall intersection point 23 that are difficult to treat with conventional applications and products, while at the same time, providing an effective treatment against viruses, molds, spores and bacteria. Finally, as the solution 34 is in the form of a liquid, transportation of the solution 34 between several locations is readily accomplished.

The foregoing detailed description of the invention has been provided for the purposes of illustration and description. Although an exemplary embodiment of the present invention has been described in detail herein with reference to the accompanying drawings, it is to be understood that the invention is not limited to the exact embodiment(s) disclosed, and that various changes and modifications to the invention are possible in light of the above teachings. Accordingly, the scope of the present invention is to be defined by any claims appended hereto.

What is claimed is:

1. An aqueous solution, comprising:
between 0.18-0.22 percent by weight of ortho-benzyl-para chlorophenol;
between 0.22-0.26 percent by weight of ortho phenylophenol;
between 0.06-0.10 percent by weight of para-tertiary amylphenol;
between 0.14-0.16 percent by weight of isopropanol;
between 0.08-0.10 percent by weight of sodium hydroxide; and
between 0.08-0.10 percent by weight of ethylene glycol.

2. The aqueous solution of claim 1, wherein the phenols, the isopropanol, the sodium hydroxide and the ethylene glycol forms a concentrate, and the concentrate is diluted in distilled water at approximately a 1:256 ratio.

3. A contaminant removal delivery system configured to generate a fog from an aqueous solution, comprising:
a fog generator; and
a container, coupled to the fog generator, including the aqueous solution therein, the aqueous solution further including:
between 0.18-0.22 percent by weight of ortho-benzyl-para chlorophenol;
between 0.22-0.26 percent by weight of ortho phenylophenol;
between 0.06-0.10 percent by weight of para-tertiary amylphenol;
between 0.14-0.16 percent by weight of isopropanol;
between 0.08-0.10 percent by weight of sodium hydroxide; and
between 0.08-0.10 percent by weight of ethylene glycol.

4. The contaminant removal delivery system of claim 3, further including a heating, ventilation and air conditioning system, coupled to the fog generator, operative to deliver the fog to within a structure.

5. The contaminant removal delivery system of claim 3, wherein the aqueous solution is diluted with distilled water from a concentrate at a ration of about one-half ounce per one gallon of water.

6. The contaminant removal delivery system of claim 3, wherein the phenols, the isopropanol, the sodium hydroxide and the ethylene glycol forms a concentrate, and the concentrate is diluted in distilled water at approximately a 1:256 ratio.
